(19) United States
(12) Patent Application Publication
(10) Pub. No.: US 2005/0085892 A1
(43) Pub. Date: Apr. 21, 2005

(54) Stent delivery device

(75) Inventors: Hiroaki Goto, Hachioji-shi (JP); Takaaki Komiya, Hachioji-shi (JP); Yoshimitsu Inoue, Hino-shi (JP)

Correspondence Address: SCULLY SCOTT MURPHY & PRESSER, PC, 400 GARDEN CITY PLAZA, GARDEN CITY, NY, 11530, US

(73) Assignee: OLYMPUS CORPORATION, TOKYO (JP)

(21) Appl. No.: 10/977,650

(22) Filed: Oct. 29, 2004

Related U.S. Application Data
(63) Continuation of application No. PCT/JP03/05581, filed on May 1, 2003.

(30) Foreign Application Priority Data
May 1, 2002 (JP) 2002-129961
May 8, 2002 (JP) 2002-133127

(51) Int. Cl. A61F 2/06

(52) U.S. Cl. 623/1.12

(57) ABSTRACT

A stent delivery device includes a stent, a guide member, a pulling member including an engaging portion that is inserted in a gap between an inner cavity of the stent and the guide member and detachably engages the stent, and an insertion portion that passes through an inner cavity of the guide member, the pulling member executing an operation for pulling the stent when the engaging portion engages the stent, and an engagement-releasing member that releases engagement between the engaging portion and the stent.

### CROSS-REFERENCE TO RELATED APPLICATIONS

This is a Continuation Application of PCT Application No. PCT/JP03/05581, filed May 1, 2003, which was published under PCT Article 21(2) in Japanese.

This application is based upon and claims the benefit of priority from prior Japanese Patent Applications No. 2002-129961, filed May 1, 2002; and No. 2002-133127, May 8, 2002, the entire contents of both of which are incorporated herein by reference.

### BACKGROUND OF THE INVENTION

1. Field of the Invention

The present invention relates to a stent delivery device that is used at a time of performing an operation for inserting a stent into a body cavity of a patient using an endoscope and positioning it there.

2. Description of the Related Art

For example, the following treatment is performed to exhaust bile, etc., which is present in the bile duct. A stent is guided to a stenotic part of the bile duct through a channel of an endoscope. Thus, the stent is positioned in the stenotic part. In this state, the bile in the bile duct is exhausted via an inner cavity of the stent.

The stent is a relatively soft hollow tube that is formed of a high-polymer compound such as polyethylene or silicone rubber, as disclosed in Jpn. U.M. Appln. KOKAI Publication No. 63-20854 (Patent Document 1). Outer peripheral portions at both ends of the stent are provided with mutually opposed flaps for preventing removal.

A therapy technique for guiding the stent with the above-described structure into a body cavity through an endoscope and positioning the stent in a stenotic part of the bile duct is performed as follows. As is shown in FIG. 23A, an elongated guide wire 3 that is formed of a flexible wire is inserted in advance in a forceps channel 2 that is provided in an insertion portion 4 of an endoscope 1. In this state, the guide wire 3, together with the insertion portion 4 of endoscope 1, is guided into a bile duct 5.

Next, the guide wire 3 is advanced and passed through the stenotic part 6 by a proximal-side manual operation. Then, as shown in FIG. 23B, using the guide wire 3, which has been passed through the stenotic part 6, as a guide, a stent 7 is pushed by a pusher tube 8 and inserted and positioned in the stenotic part 6.

However, the stenotic part 6 is located in a deep region of the body cavity. This disables direct observation of stenotic part 6 by the endoscope 1. In general, the stenotic part 6 is treated under X-ray imaging. In this case, it is likely that the stent 7 is pushed too deeply into the stenotic part 6 by the pusher tube 8. However, the stent 7 and pusher tube 8 are not coupled. If the stent 7 is pushed too deeply, the stent 7 cannot be pulled back even if the pusher tube 8 is pulled, as indicated by an arrow in FIG. 23B.

To solve this problem, a drainage catheter delivery system, as disclosed in U.S. Pat. No. 5,921,952 (Patent Document 2), has been developed. In this system, a pusher tube and a stent are coupled by a suture. When the stent is pushed too deeply, the stent can be pulled back by means of the suture if the pusher tube is pulled.

In the system of Patent Document 2, a distal end portion of the pusher tube is provided with an insertion hole for insertion of the suture. The stent is provided with an opening that is made by forming a flap. A suture that is engaged with the guide wire is led out of the opening of the stent. Then, the suture is passed through the insertion hole of the pusher tube and knotted. Thus, the stent and pusher tube are coupled.

Thus, when the stent is pushed too deeply, if the pusher tube is pulled, the stent can be pulled back by means of the suture. In addition, after the stent is positioned in the stenotic part, the guide wire is pulled back. At this time, if the distal end portion of the guide wire is disengaged from the engagement part with the suture, the suture is removed from the stent. Thereby, the stent and the pusher tube are separated.

In the system of Patent Document 2, after the stent is stayed in the stenotic part, the guide wire is pulled back. At this time, if the distal end portion of the guide wire is not disengaged from the engagement part with the suture, the stent and the pusher tube are not separated.

Thus, at the time of the procedure for positioning the stent, the guide wire is pulled off. Consequently, even if a subsequent treatment is to be performed using the guide wire as a guide after the stent is positioned, such a treatment cannot be performed.

In the system of Patent Document 2, in the setting condition prior to use, the stent is passed over the guide wire, and the distal end portion of the stent is held in contact with the distal end portion of the pusher tube. Since the coupling part between the pusher tube and stent is kept in such a state that the end faces of the pusher tube and stent are merely abutted on each other, the bending strength of the coupling part between the pusher tube and stent is weak. Consequently, when the pusher tube is advanced in the procedure for positioning the stent in the stenotic part, buckling may occur at the coupling part between the pusher tube and the stent, and the stent may not be approached to a target part.

Moreover, the distal end portion of the pusher tube needs to be provided with the insertion hole for insertion of the suture. Consequently, when a liquid is fed through the pusher tube, the liquid may disadvantageously leak from the insertion hole.

The present invention has been made in consideration of the above circumstances, and the object of the invention is to provide a stent guide that is configured such that a stent, which is pushed too deeply at a time of a procedure for positioning the stent, can be pulled back, that the stent has a high bending strength and can be advanced to a target position even when the bending angle of a curved part of an endoscope is large, and that there is no liquid leak when a liquid is fed.

### BRIEF SUMMARY OF THE INVENTION

According to the present invention, a stent delivery device included a stent, a guide member having an inner cavity, at least a distal end portion of the guide member being insertable in the stent, a pulling member including an engaging portion that is inserted in a gap between the inner cavity of the stent and the guide member and detachably engages the stent, and an insertion portion that passes through at least a part of the inner cavity of the guide member, the pulling member executing an operation for pulling the stent when the engaging portion engages the stent, and an engagement-releasing member that moves the pulling member in an axial direction of the guide member, thereby releasing the engagement between the engaging portion and the stent.

According to the above structure, since the engaging portion, which is connected to a distal end portion of the pulling member, is positioned in the engaged state between the stent and the guide member, the stent can be approached to a target part and positioned by advancing the guide member. When the stent is pushed too deeply, the stent can be pulled back by pulling the pulling member toward a proximal-end side.

After the stent is positioned at the target part, the pulling member is pulled toward the proximal-end side while the guide member is being held. Thereby, the engagement between the engaging portion and the stent can be released, and the stent can be positioned at the target part. Furthermore, since the distal end portion of the guide member is inserted through the inner cavity of the stent, the stent can be advanced to the target part in accordance with the curving of the curved part of the endoscope.

### BRIEF DESCRIPTION OF THE SEVERAL VIEWS OF THE DRAWING

FIG. 1A is a side view showing the entirety of a stent delivery device according to a first embodiment of the present invention;

FIG. 1B is a longitudinal cross-sectional view of a distal end portion of the stent delivery device according to the first embodiment;

FIG. 2A is a partially cut-out side view of a stent delivery device according to a second embodiment of the present invention;

FIG. 2B is a cross-sectional view taken along line IIB-IIB in FIG. 2A;

FIG. 2C is a cross-sectional view taken along line IIC-IIC in FIG. 2A;

FIG. 2D is a side view of the stent delivery device according to the second embodiment;

FIG. 3A is a longitudinal cross-sectional view of a distal end portion of a stent delivery device according to a third embodiment of the invention;

FIG. 3B is a side view of the distal end portion of the stent delivery device shown in FIG. 3A;

FIG. 4A is a partially cut-out side view of a stent delivery device according to a fourth embodiment of the present invention;

FIG. 4B is a cross-sectional view taken along line IVB-IVB in FIG. 4A;

FIG. 4C is a cross-sectional view taken along line IVC-IVC in FIG. 4A;

FIG. 4D is a side view of the stent delivery device according to the fourth embodiment;

FIG. 5A is a partially cut-out side view of the stent delivery device according to the fourth embodiment, showing the state in which a flexible wire and a stent are separated;

FIG. 5B is a side view of the stent delivery device according to the fourth embodiment, showing the state in which the flexible wire and the stent are separated;

FIG. 6A is a longitudinal cross-sectional view of the distal end portion of the stent delivery device according to the fourth embodiment, showing the state in which the stent is positioned in a stenotic part by the stent delivery device;

FIG. 6B is a longitudinal cross-sectional view of a main part of a modification of the stent delivery device according to the fourth embodiment;

FIG. 6C is a longitudinal cross-sectional view showing the state in which the flexible wire of the stent delivery device shown in FIG. 6B is pushed in;

FIG. 7 is a side view showing a stent delivery device according to a fifth embodiment of the present invention;

FIG. 8A is a longitudinal cross-sectional view of a distal end portion of a stent delivery device according to a sixth embodiment of the present invention;

FIG. 8B is a longitudinal cross-sectional view of a main part of a first modification of the stent delivery device according to the sixth embodiment;

FIG. 8C is a longitudinal cross-sectional view of a main part of a second modification of the stent delivery device according to the sixth embodiment;

FIG. 8D is a longitudinal cross-sectional view of a main part of a third modification of the stent delivery device according to the sixth embodiment;

FIG. 8E is a longitudinal cross-sectional view of a main part of a fourth modification of the stent delivery device according to the sixth embodiment;

FIG. 8F is a longitudinal cross-sectional view of a main part of a fifth modification of the stent delivery device according to the sixth embodiment;

FIG. 8G is a longitudinal cross-sectional view of a main part of a sixth modification of the stent delivery device according to the sixth embodiment;

FIG. 8H is a longitudinal cross-sectional view of a main part of a seventh modification of the stent delivery device according to the sixth embodiment;

FIG. 8I is a longitudinal cross-sectional view of a main part of an eighth modification of the stent delivery device according to the sixth embodiment;

FIG. 9 is a partially cut-out side view of a stent delivery device according to a seventh embodiment of the present invention;

FIG. 10 is a plan view showing the state in which the stent and the engaging member of the stent delivery device according to the seventh embodiment are engaged;

FIG. 11A is a longitudinal cross-sectional view showing the state in which the engaging member of the stent delivery device according to the seventh embodiment is set in a position of engagement with the stent;

FIG. 11B is a longitudinal cross-sectional view showing the state in which the engaging member of the stent delivery device according to the seventh embodiment is pushed forward and disengaged from the stent;

FIG. 11C is a longitudinal cross-sectional view showing the state in which the engaging member of the stent delivery device according to the seventh embodiment is pulled to the proximal-end side;

FIG. 12 is an explanatory view for explaining the operation state of the engaging member of the stent delivery device according to the seventh embodiment;

FIG. 13A is a perspective view showing the state in which the stent and pusher tube of a stent delivery device according to an eighth embodiment of the invention are connected;

FIG. 13B is a perspective view showing the state in which the stent and pusher tube of the stent delivery device according to the eighth embodiment are separated;

FIG. 13C is a longitudinal cross-sectional view of a part A in FIG. 13A;

FIG. 14 is a longitudinal cross-sectional view of a connection part between the stent and pusher tube according to a ninth embodiment of the invention;

FIG. 15 is a longitudinal cross-sectional view of a connection part between the stent and pusher tube according to a tenth embodiment of the invention;

FIG. 16A is a perspective view showing the state in which the stent and pusher tube of a stent delivery device according to an eleventh embodiment of the invention are connected;

FIG. 16B is a perspective view showing the state in which the stent and pusher tube of the stent delivery device according to the eleventh embodiment are separated;

FIG. 16C is a longitudinal cross-sectional view of a part B in FIG. 16A;

FIG. 17A is a transverse cross-sectional view showing the state in which a cylindrical member of an apparatus according to a twelfth embodiment of the invention is press-fitted in inner cavities of the stent and pusher tube;

FIG. 17B is a transverse cross-sectional view showing a modification of the stent of the twelfth embodiment;

FIG. 18 is a perspective view showing the state in which the stent and pusher tube of an apparatus according to a 13th embodiment of the invention are connected;

FIG. 19 is a longitudinal cross-sectional view of a part B in FIG. 18;

FIG. 20 is a perspective view showing the state in which the stent and pusher tube of the apparatus according to the 13th embodiment are separated;

FIG. 21A is a longitudinal cross-sectional view of a main part of an apparatus according to a 14th embodiment of the invention in the state in which the stent and pusher tube are connected;

FIG. 21B is a longitudinal cross-sectional view showing a main part of the apparatus according to the 14th embodiment in the state in which the stent and pusher tube are separated;

FIG. 22A is a longitudinal cross-sectional view of a main part of a modification of the 14th embodiment in the state in which the stent and pusher tube are connected;

FIG. 22B is a longitudinal cross-sectional view showing the main part in the state in which the stent and pusher tube shown in FIG. 22A are separated;

FIG. 23A is an explanatory view for explaining a therapy technique for guiding a stent into a body cavity through an endoscope; and

FIG. 23B is an explanatory view for explaining the state in which the stent is pushed in by a pusher tube, inserted in a stenotic part, and positioned.

### DETAILED DESCRIPTION OF THE INVENTION

Embodiments of the present invention will now be described with reference to the accompanying drawings.

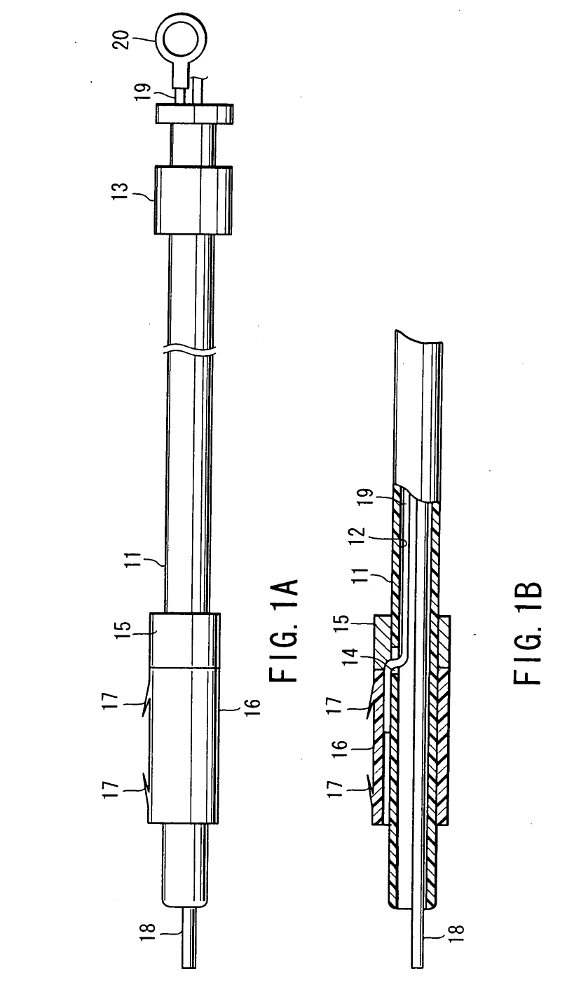

FIG. 1A and FIG. 1B show a stent delivery device according to a first embodiment.

As is shown in FIG. 1A, the stent delivery device according to the present embodiment is provided with an elongated guide catheter 11 that is insertable in a forceps channel of an endoscope (not shown). The guide catheter 11 is formed of a flexible synthetic resin material such as a fluoro-resin or a nylon resin. An inner cavity 12 is formed in the guide catheter 11 over the entire length thereof. A guide catheter cock 13 is provided near a proximal end portion of the guide catheter 11.

As is shown in FIG. 1B, a single small hole 14 is formed in a side wall of the guide catheter 11 near a distal end portion of the guide catheter 11. A fixing ring 15, which is an enlarged part with a large outside diameter, is fitted on the outer peripheral surface of the guide catheter 11 at a position corresponding to the small hole 14. The fixing ring 15 is disposed so as to close part of the small hole 14.

In addition, a hollow-tube-like stent 16, which serves as a stent, is provided on the outer peripheral surface of the guide catheter 11 on the distal-end side of the fixing ring 15. In the state in which the stent 16 is engaged with the guide catheter 11, the small hole 14 of the guide catheter 11 is closed by the fixing ring 15 and the stent 16.

The stent 16 is formed of a resin with biocompatibility, such as polyethylene, fluoro-resin, nylon resin, thermoplastic elastomer or silicone rubber. It is desirable that the outer peripheral surface of the stent 16 be coated with a hydrophilic lubricant. Outer peripheral portions at both ends of the stent 16 are provided with mutually opposed flaps 17 for preventing removal.

A guide wire 18 and a flexible wire 19 serving as a pulling member are passed through the inner cavity 12 of the guide catheter 11. The flexible wire 19 is formed of an elongated metallic twisted wire. The flexible wire 19 may partly be formed of a fibrous member of, e.g. stainless steel, nickel, a titanium alloy, nylon, liquid crystal polymer, or silk. The flexible wire 19 may have a substantially rectangular cross section or a substantially circular cross section. Further, the flexible wire 19 may be provided with a large-diameter portion at a distal end thereof.

The guide wire 18 is formed of an elongated metallic linear material, twisted material or coil-shaped material. The guide wire 19 should preferably be formed of a metal with superelastic properties of, in particular, a nickel-titanium alloy. The distal end portion of the guide wire 18 is tapered. Further, the proximal end portion of the guide wire 18 is led out of the guide catheter cock 13. The distal end side of the flexible wire 19 is led out of the guide catheter 11 from the inner cavity of the guide catheter 11 via the small hole 14.

The distal end portion of the flexible wire 19 is press-fitted between the inner peripheral surface of the stent 16 and the outer peripheral surface of the guide catheter 11. An operation ring 20 is provided at the proximal end of the flexible wire 19.

The distal end portion of the flexible wire 19 is not necessarily press-fitted over the entire length of the stent 16. It should suffice if the distal end portion of the flexible wire 19 is pressed-fitted over such a length that the stent 16 can be moved together with the flexible wire 19 toward the proximal end side when the flexible wire 19 is pulled toward the proximal end side. For example, a portion of the flexible wire 19, which has an axial length of about 5 mm or more, may be positioned within the stent 16.

If the flexible wire 19 is pulled to the proximal end side with a greater force in the state in which the stent 16 abuts on the fixing ring 15, the engagement between the stent 16 and flexible wire 19 is released and the stent 16 is separated from the flexible wire 19.

Next, the operation of the first embodiment is described. When the stent delivery device according to this embodiment is used, the stent delivery device is set as follows.

To begin with, the flexible wire 19 is inserted in the inner cavity 12 of the guide catheter 11 of the stent delivery device. The distal end portion of the flexible wire 19 is led out of the small hole 14. Then, the stent 16 is fitted on the guide catheter 11 from the distal end thereof. Further, the distal end portion of the flexible wire 19 is press-fitted between the stent 16 and guide catheter 11. Thus, as shown in FIG. 1B, the guide catheter 11, stent 16 and flexible wire 19 are set in the assembled state.

The insertion portion of the endoscope is inserted in a body cavity in advance, and a distal-end structural part, which is disposed at the distal end of the insertion portion of the endoscope, is guided to the vicinity of the bile duct.

Subsequently, the guide wire 18 is passed through the forceps channel of the endoscope that is inserted in the body. At this time, under observation using the endoscope and observation using X-rays, the guide wire 18 is advanced and the distal end portion of the guide wire 18 is guided into a stenotic part of the bile duct.

Thereafter, as described above, the guide catheter 11, on which the stent 16 is set, is passed over the guide wire 18, and the guide catheter 11 is inserted into the forceps while being guided by the guide wire 18.

At this time, the guide catheter 11 is advanced by a manual operation on the proximal end side of the guide catheter 11. The guide catheter 11 is led out of the distal-end structural part of the endoscope, and the guide catheter 11 and stent 16 are inserted into the stenotic part.

During the operation for inserting the guide catheter 11, the stent 16 is kept fitted on the guide catheter 11. Thus, the bending strength of the stent 16 is high, and even if the guide wire 18 is curved with a large angle of bend, the stent 16 is not buckled. The stent 16 can be advanced together with the guide catheter 11 and guided to a target part.

After the stent 16 is inserted into the stenotic part by means of the guide catheter 11, X-ray observation is performed. If it is confirmed by the X-ray observation that the stent 16 is pushed too deeply, an operation for pulling back the stent 16 to a position on the proximal end side is performed. In the procedure, the finger is hooked on the operation ring 20 and pulled to the proximal end side. Thereby, the stent 16 can be pulled back by the flexible wire 19, and the stent 16 can exactly be positioned at the target part.

Then, an operation for pulling the flexible 19 toward the proximal end side is performed by hooking the finger on the operation ring 20 while holding the guide catheter 11. In this operation, the distal end portion of the flexible wire 19 is removed from between the guide catheter 11 and stent 16. As a result, the flexible wire 19 and stent 16 are separated, and the stent 16 is positioned at the stenotic part.

In this case, the guide wire 18 is kept in the state in which the guide wire 18 is passed through the guide catheter 11. The distal end portion of the guide wire 18 is left at the position of the stenotic part. Thus, using the guide wire 18 as a guide, a subsequent treatment may be performed.

In addition, since the guide catheter 11 has the inner cavity 12, it is possible to feed or suck a liquid from the guide catheter cock 13.

The apparatus with the above structure can achieve the following advantageous effects. In the stent delivery device according to the present embodiment, the stent 16 is fitted on the guide catheter 11, and the distal end portion of the flexible wire 19 is press-fitted between the stent 6 and guide catheter 11. Thereby, as shown in FIG. 1B, the guide catheter 11, stent 16 and flexible wire 19 are set in the integrally assembled state. Therefore, at the time of the procedure for positioning the stent 16 at the stenotic part of the bile duct, the stent 16 can be pulled back to the proximal end side by means of the flexible wire 19 even if the stent 16 is pushed too deeply.

Moreover, during the work for inserting the guide catheter 11, the stent 16 is kept fitted on the guide catheter 11. Thus, the bending strength of the stent 16 is high, and even if the angle of bend of the curved part of the endoscope is large, the stent 16 can be advanced to the target part.

Furthermore, the small hole 14 in the guide catheter 11 is closed by the fixing ring 15 and stent 16. Therefore, when a liquid is fed, there is no possibility of liquid leak from the small hole 14.

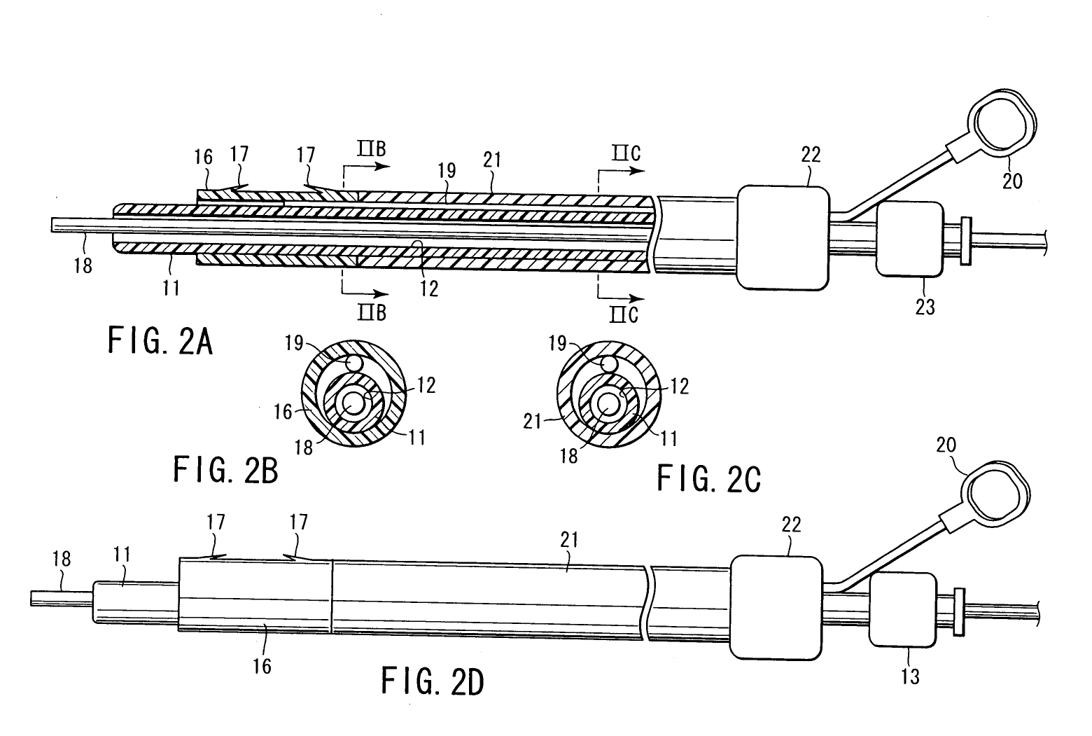

FIGS. 2A to 2D show a second embodiment of the present invention. The structural parts common to those in the first embodiment are denoted by like reference numerals, and a description thereof is omitted. The stent 16 is fitted on the distal end portion of the guide catheter 11.

In addition, a pusher tube 21 is axially movably fitted on the outer peripheral surface of the guide catheter 11 on the proximal end side of the stent 16. The pusher tube 21 is formed of a flexible synthetic resin material. A pusher tube cock 22 is provided at the proximal end of the pusher tube 21.

A flexible wire 19, which serves as a pulling member, is axially movably passed between the outer peripheral surface of the guide catheter 11 and the inner peripheral surface of the pusher tube 21. A distal end portion of the flexible wire 19 is press-fitted between the inner peripheral surface of the stent 16 and the outer peripheral surface of the guide catheter 11.

A proximal end portion of the flexible wire 19 is led out of the pusher tube cock 22. An operation ring 20 is provided at the proximal end of the flexible wire 19.

Next, the operation of the stent delivery device according to the second embodiment is described. When the stent delivery device according to this embodiment is used, the stent delivery device is set as follows.

To begin with, the flexible wire 19 is passed between the guide catheter 11 and the pusher tube 21 of the stent delivery device. Then, the stent 16 is fitted on the distal end portion of the guide catheter 11. Subsequently, the distal end portion of the flexible wire 19 is press-fitted between the stent 16 and guide catheter 11. Thus, as shown in FIG. 2A, the guide catheter 11, stent 16, flexible wire 19 and pusher tube 21 are set in the assembled state.

Thereafter, like the first embodiment, the guide wire 18 is passed through the forceps channel of the endoscope. Then, the operation for guiding the stent 16 to the stenotic part of the bile duct by means of the guide catheter 11 is performed. This method is the same as in the first embodiment. In the present embodiment, the stent 16 is inserted into the stenotic part by advancing the pusher tube 21.

After the stent 16 is inserted into the stenotic part by means of the pusher tube 21, X-ray observation is performed. If it is confirmed by the X-ray observation that the stent 16 is pushed too deeply, an operation for pulling back the stent 16 to a position on the proximal end side is performed. In the procedure, the finger is hooked on the operation ring 20 and pulled to the proximal end side. Thereby, the stent 16 can be pulled back by the flexible wire 19, and the stent 16 can exactly be positioned at the target part.

Then, an operation for pulling the flexible 19 toward the proximal end side is performed by hooking he finger on the operation ring 20 while holding the guide catheter 11. In this operation, the distal end portion of the flexible wire 19 is removed from between the guide catheter 11 and stent 16. As a result, the flexible wire 19 and stent 16 are separated, and the stent 16 is stayed at the stenotic part.

In this case, the guide wire 18 is kept in the state in which the guide wire 18 is passed through the guide catheter 11. The distal end portion of the guide wire 18 is left at the position of the stenotic part. Thus, using the guide wire 18 as a guide, a subsequent treatment may be performed.

In addition, since the guide catheter 11 has the inner cavity 12, it is possible to feed or suck a liquid from the guide catheter cock 13.

In the apparatus with the above structure, too, at the time of the procedure for positioning the stent 16 at the stenotic part of the bile duct, the stent 16 can be pulled back to the proximal end side by means of the flexible wire 19 even if the stent 16 is pushed too deeply.

Moreover, during the work for inserting the guide catheter 11, the stent 16 is kept engaged with the guide catheter 11. In addition, the distal end portion of the flexible wire 19 is inserted in the inner cavity of the stent 16. Thus, the bending strength of the stent 16 is high. Even if the angle of bend of the curved part of the endoscope is large, the stent 16 can be advanced to the target part in accordance with the curving of the curved part of the endoscope.

After the stent 16 is positioned at the target part, the flexible wire 19 is pulled to the proximal end side while the guide catheter 11 is being held. Thereby, the engagement between the distal end portion of the flexible wire 19 and the stent 16 is released. Thus, the stent 16 can be positioned at the target part.

Furthermore, when a liquid is fed through the guide catheter 11, there is no possibility of liquid leak.

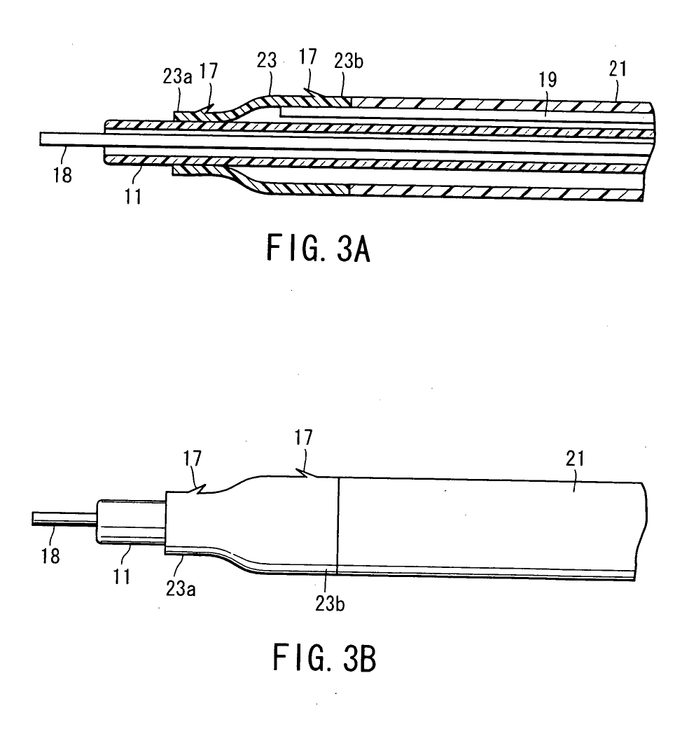

FIG. 3A and FIG. 3B show a third embodiment of the present invention. The structural parts common to those in the first embodiment are denoted by like reference numerals, and a description thereof is omitted.

A stent 23 of this embodiment has a small-diameter portion 23a at a distal end thereof. The stent 23 has a large-diameter portion 23b at a proximal end thereof. The large-diameter portion 23b has the same diameter as the pusher tube 21.

The distal end portion of the flexible wire 19 is press-fitted between the inner peripheral surface of the large-diameter portion 23b of the stent 23 and the outer peripheral surface of the guide catheter 11.

Since the stent 23 of this embodiment has the small-diameter portion 23a at its distal end, the stent 23 can easily be inserted into the stenotic part of the bile duct.

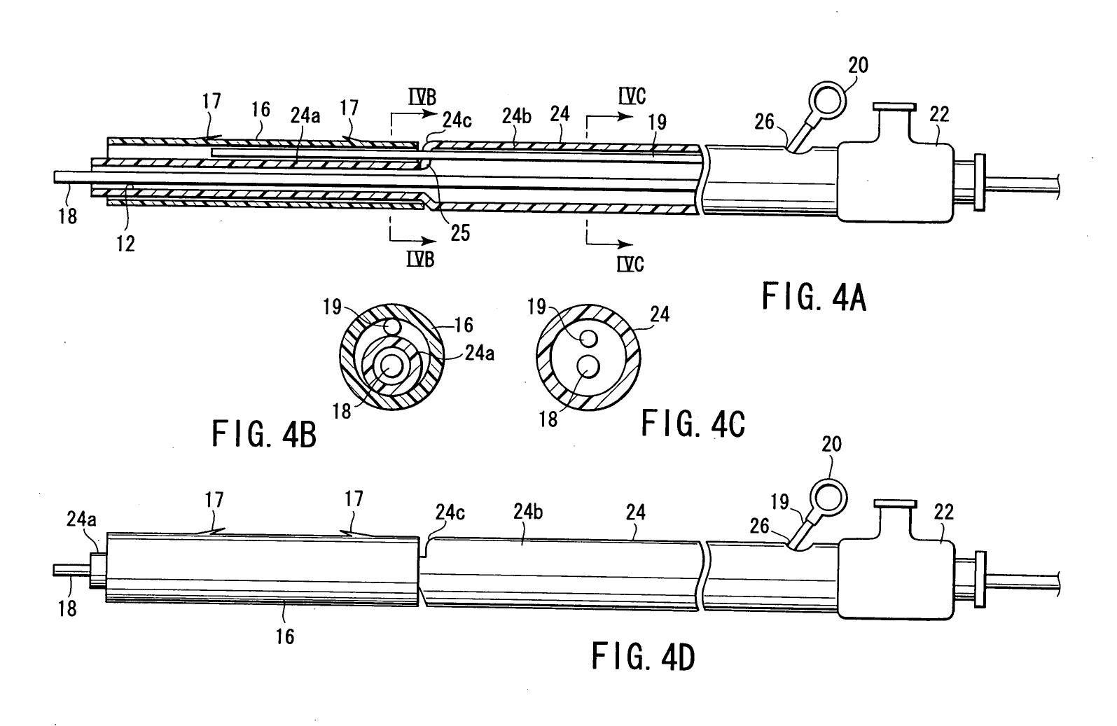

FIG. 4A to FIG. 6A show a fourth embodiment of the present invention. In this embodiment, a pusher tube 24, which has a structure different from the structure of the pusher tube 21 of the second embodiment (see FIGS. 2A to 2D), is provided. The other structural parts are the same as those in the second embodiment. The structural parts common to those in the second embodiment are denoted by like reference numerals, and a description thereof is omitted.

As is shown in FIG. 4A, in this embodiment, the pusher tube 24 includes a small-diameter portion 24a at a distal end thereof. The small-diameter portion 24a is inserted in the inner cavity of the stent 16.

The pusher tube 24 is provided with a stepped portion 24c between the small-diameter portion 24a and a large-diameter portion 24b thereof, which is formed on the proximal end side of the small-diameter portion 24a. The stepped portion 24c has a passage hole 25.

The distal end portion of the flexible wire 19, which is passed through the inner cavity of the pusher tube 24, is led out of the passage hole 25 to the outside of the small-diameter portion 24a. The distal end portion of the flexible wire 19 is press-fitted between the inner peripheral surface of the stent 16 and the outer peripheral surface of the small-diameter portion 24a.

Further, a side hole 26 is formed in the side wall of the pusher tube 24. The proximal end portion of the flexible wire 19 is led out of the side hole 26 of the pusher tube 24.

Next, the operation of the fourth embodiment is described. When the stent delivery device according to this embodiment is used, the stent delivery device is set as follows.

To begin with, the flexible wire 19 is inserted in the inner cavity of the pusher tube 24, and the distal end portion thereof is led out of the passage hole 25 to the outside of the small-diameter portion 24a. Then, the stent 16 is fitted on the distal-end small-diameter portion 24a of the pusher tube 24. Further, as shown in FIG. 4B, the distal end portion of the flexible wire 19 is press-fitted between the inner peripheral surface of the stent 16 and the outer peripheral surface of the small-diameter portion 24a. Thus, as shown in FIG. 4A, the pusher tube 24, stent 16 and flexible wire 19 are set in the assembled state.

The guide wire 18 is passed through the forceps channel of the endoscope, and the stent 16 is guided to the stenotic part of the bile duct by means of the pusher tube 24 in the same manner as in the first embodiment. In the present embodiment, when the pusher tube 24 is advanced, the stepped portion 24c abuts on the proximal end of the stent 16. If the pusher tube 24 is further advanced, the stent 16 is inserted into the stenotic part.

After the stent 16 is inserted into the stenotic part by means of the pusher tube 24, X-ray observation is performed. If it is confirmed by the X-ray observation that the stent 16 is pushed too deeply, an operation for pulling back the stent 16 to a position on the proximal end side is performed. In the procedure, the finger is hooked on the operation ring 20 and pulled to the proximal end side. Thereby, the stent 16 can be pulled back by the flexible wire 19, and the stent 16 can exactly be positioned at the target part.

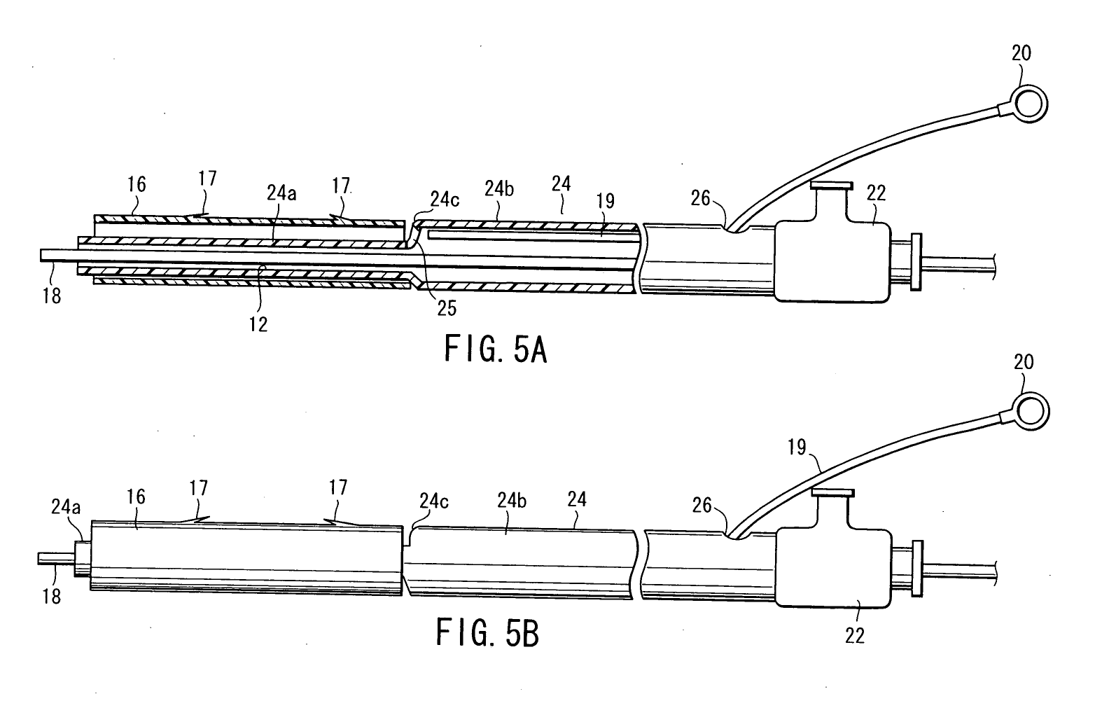

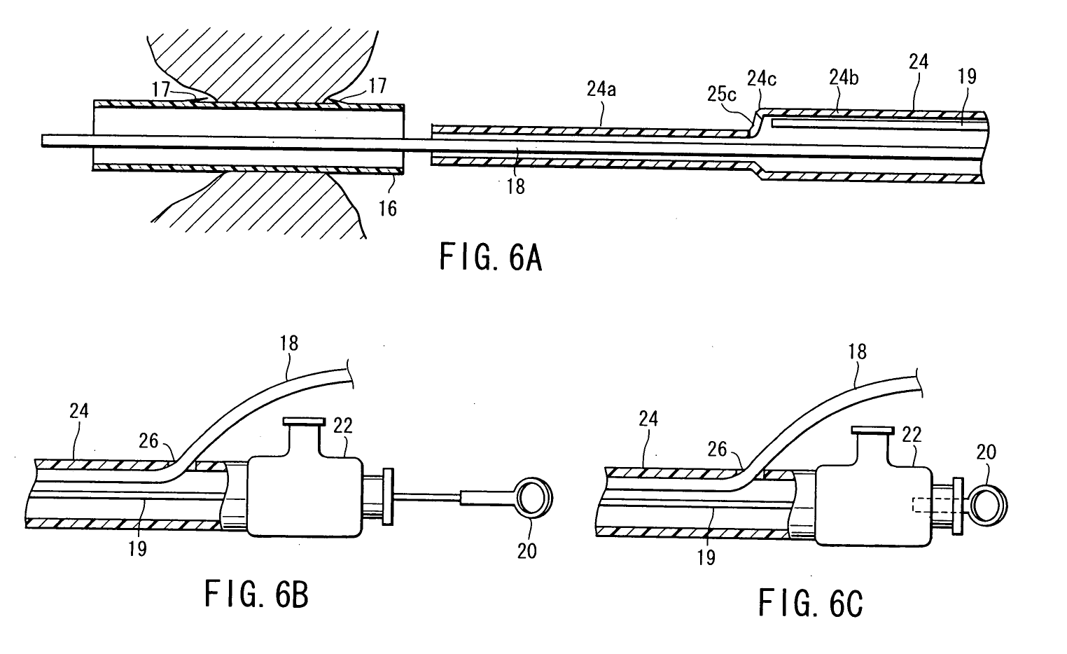

Then, an operation for pulling the flexible 19 toward the proximal end side is performed by hooking the finger on the operation ring 20 while holding the pusher tube 24. In this operation, as shown in FIG. 5A, the distal end portion of the flexible wire 19 is removed from between the pusher tube 24 and stent 16. As a result, as shown in FIG. 6A, the flexible wire 19 and stent 16 are separated. The stent 16 is thus positioned at the stenotic part.

In this case, the guide wire 18 is kept in the state in which the guide wire 18 is passed through the pusher tube 24. The distal end portion of the guide wire 18 is left at the position of the stenotic part. Thus, using the guide wire 18 as a guide, a subsequent treatment may be performed.

In the apparatus with the above structure, too, at the time of the procedure for positioning the stent 16 at the stenotic part of the bile duct, the stent 16 can be pulled back to the proximal end side by means of the flexible wire 19 even if the stent 16 is pushed too deeply.

In this embodiment, the guide catheter 11 can be dispensed with, so cost reduction is possible. Further, the diameter of the stent 16 and pusher tube 24 can be reduced. In a case where the stenotic part is small in size, the stent 16 can easily be inserted.

FIG. 6B and FIG. 6C show a modification of the stent delivery device of the fourth embodiment. The guide wire 18 is inserted into the pusher tube 24 via the side hole 26 of the pusher tube 24.

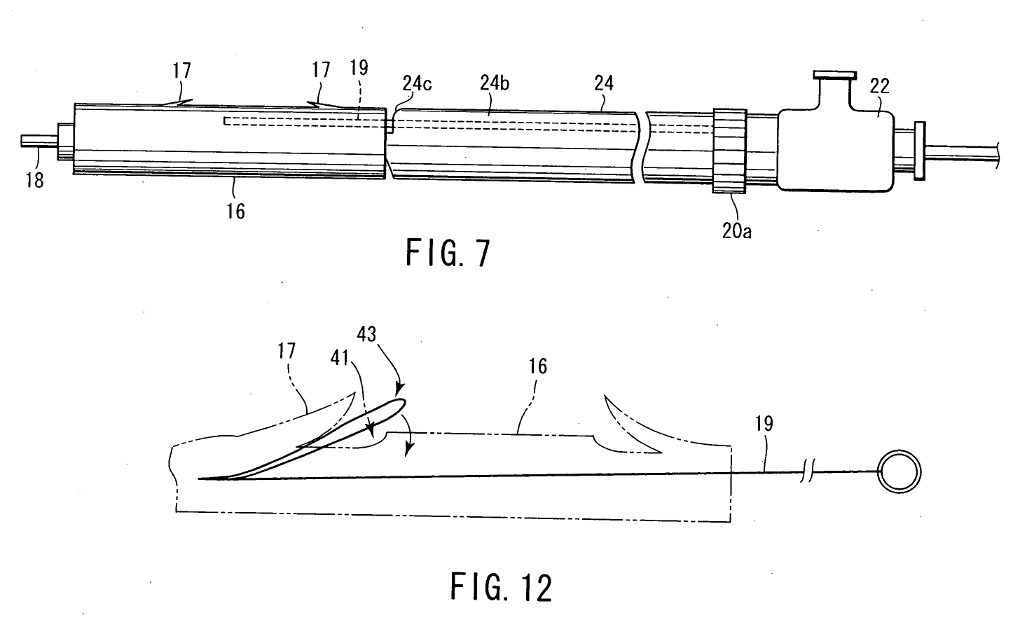

FIG. 7 shows a fifth embodiment of the invention. In this embodiment, the stent delivery device of the fourth embodiment (see FIG. 4A through FIG. 6C) is modified as follows. The parts common to those in the fourth embodiment are denoted by like reference numerals, and a description thereof is omitted.

In this embodiment, an operation ring 20a is axially movably fitted on a proximal end portion of the guide catheter 11. The proximal end of the flexible wire 19, which is inserted in the pusher tube 24, is coupled to the operation ring 20a.

According to this embodiment, the flexible wire 19 is pulled by moving the operation ring 20a backward. Thereby, the engagement between the flexible wire 19 and stent 16 can be released.

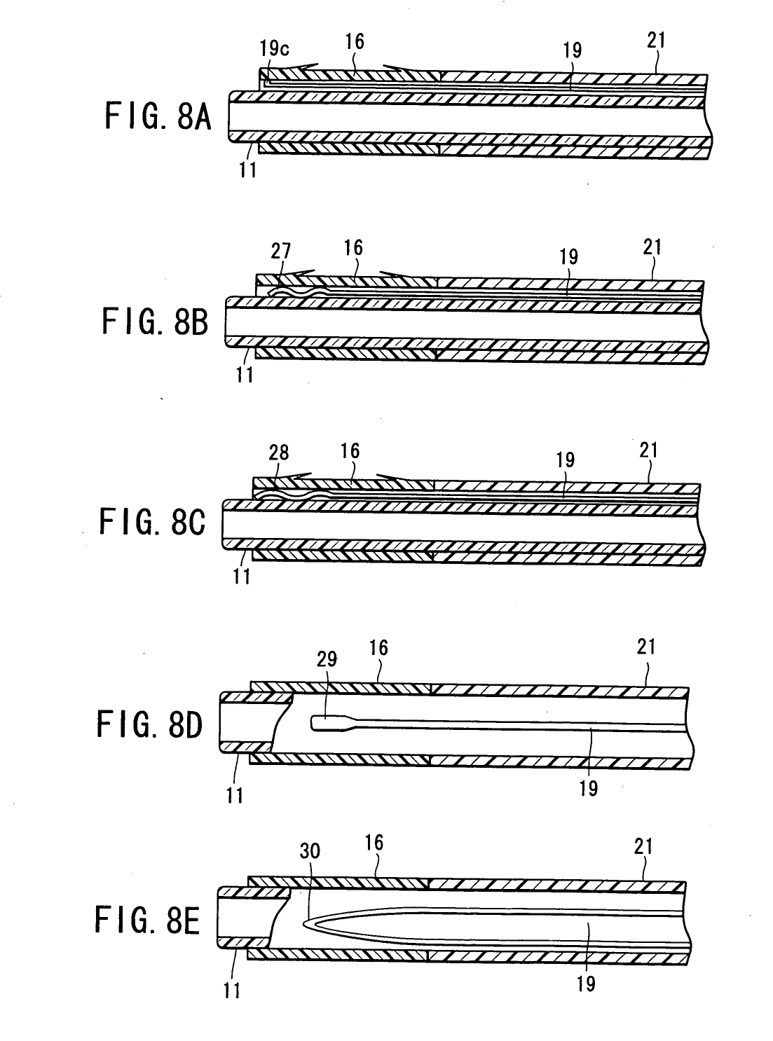

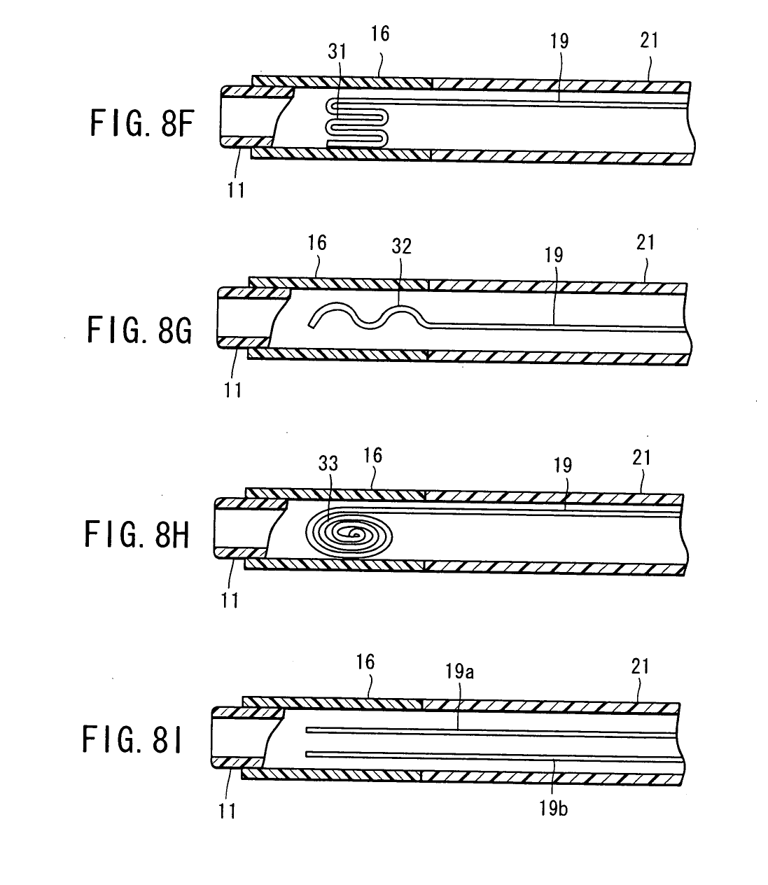

FIG. 8A shows a sixth embodiment of the invention. In this embodiment, the parts common to those in the second embodiment (see FIGS. 2A to 2D) are denoted by like reference numerals, and a description thereof is omitted. In this embodiment, the distal end portion of the flexible wire 19 is provided with a bent portion 19c that is bent upward. The bent portion 19c is put in pressure contact with the inner wall of the stent 16.

FIG. 8B shows a first modification of the stent delivery device according to the sixth embodiment. In this modification, the distal end portion of the flexible wire 19 is provided with a wavy bent portion 27. The wavy bent portion 27 is put in pressure contact with the inner wall of the stent 16.

FIG. 8C shows a second modification of the stent delivery device according to the sixth embodiment. In this modification, the distal end portion of the flexible wire 19 is provided with an S-shaped bent portion 28. The S-shaped bent portion 28 is put in pressure contact with the inner wall of the stent 16.

FIG. 8D shows a third modification of the stent delivery device according to the sixth embodiment. In this modification, the distal end portion of the flexible wire 19 is provided with a widened portion 29 with an increased dimension in its width direction. The widened portion 29 is put in pressure contact with the inner wall of the stent 16.

FIG. 8E shows a fourth modification of the stent delivery device according to the sixth embodiment. In this modification, a single flexible wire 19 is folded within the stent 16 and thus provided with a folded portion 30. The folded portion 30 is put in pressure contact with the inner wall of the stent 16.

FIG. 8F shows a fifth modification of the stent delivery device according to the sixth embodiment. In this modification, the distal end portion of the flexible wire 19 is provided with a meandering portion 31. The meandering portion 31 is put in pressure contact with the inner wall of the stent 16.

FIG. 8G shows a sixth modification of the stent delivery device according to the sixth embodiment. In this modification, the distal end portion of the flexible wire 19 is provided with a wavy bent portion 32 that is bent in a wavy shape in the width direction. The wavy bent portion 32 is put in pressure contact with the inner wall of the stent 16.

FIG. 8H shows a seventh modification of the stent delivery device according to the sixth embodiment. In this modification, the distal end portion of the flexible wire 19 is provided with a spiral portion 33. The spiral portion 33 is put in pressure contact with the inner wall of the stent 16.

FIG. 8I shows a first modification of the stent delivery device according to the sixth embodiment. In this modification, two flexible wires 19a and 19b are provided in parallel. The distal end portions of the two flexible wires 19a and 19b are put in pressure contact with the inner wall of the stent 16.

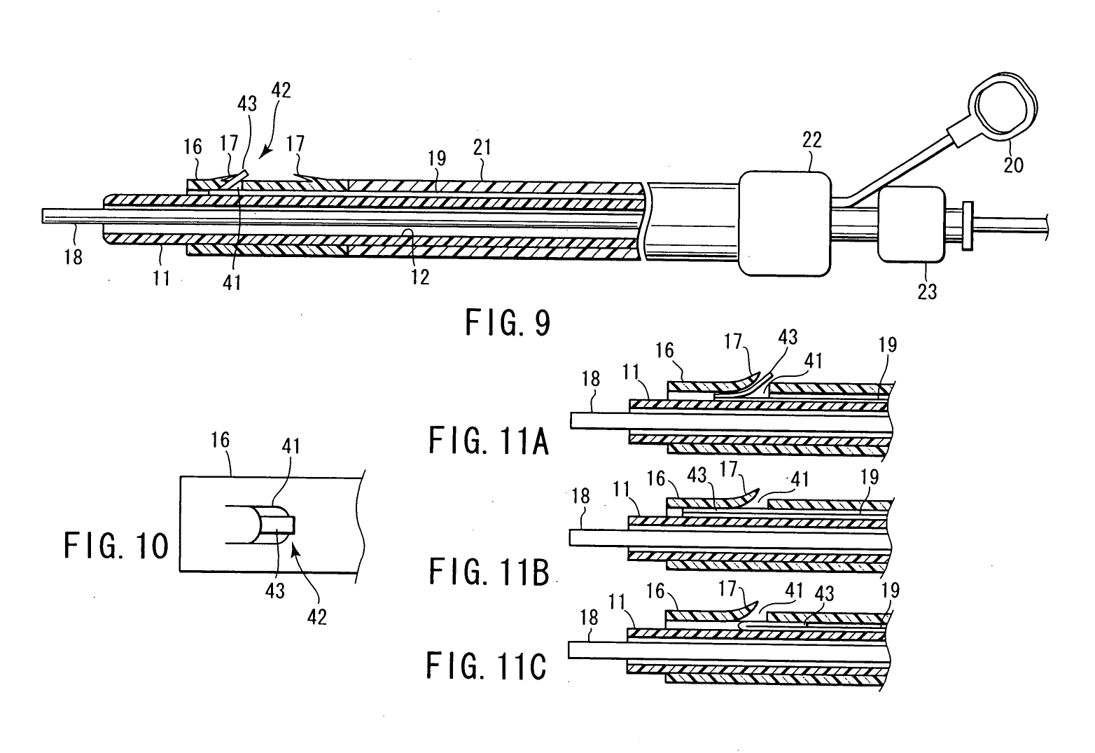

FIG. 9 to FIG. 12 show a seventh embodiment of the present invention. In this embodiment, the stent delivery device of the second embodiment (see FIG. 2A through FIG. 2D) is modified as follows. As regards the stent delivery device of this embodiment, the parts common to those in the second embodiment are denoted by like reference numerals, and a description thereof is omitted.

As is shown in FIG. 9, a stent 16 of the stent delivery device of the present embodiment is provided with an opening 117a at a distal end side thereof, which is made by forming a flap 17.

A distal end portion of the flexible wire 19, which serves as a pulling member, is provided with an engaging member 42. The engaging member 42 is provided with a plate-spring-like engaging plate 43. The engaging plate 43 has spring characteristics and is formed using a plate-like member that is made of a spring material of, e.g. stainless steel.

A front end portion of the engaging plate 43 is fixed to a distal end portion of the flexible wire 19 by means of brazing. As is shown in FIG. 9 and FIG. 10, a rear end portion of the engaging plate 43 is inserted in a side hole 17a that is made by the flap 17 of the stent 16, and is detachably hooked.

As is shown in FIG. 11A and FIG. 12, the stent delivery device of this embodiment is set in the state in which the rear end portion of the engaging plate 43 is inserted and hooked in the side hole 17a of the stent 16. In this state, like the first embodiment, the guide wire 18 is passed through the forceps channel of the endoscope, and the stent 16 is guided to the stenotic part of the bile duct by means of the guide catheter 11 in the same manner as in the first embodiment. In the present embodiment, the stent 16 is inserted into the stenotic part 16 by advancing the pusher tube 21.

After the stent 16 is inserted into the stenotic part by means of the pusher tube 21, X-ray observation is performed. If it is confirmed by the X-ray observation that the stent 16 is pushed too deeply, an operation for pulling back the stent 16 to a position on the proximal end side is performed. In the procedure, the finger is hooked on the operation ring 20 and pulled to the proximal end side. Thereby, the stent 16 can be pulled back by the flexible wire 19, and the stent 16 can exactly be positioned at the target part.

When the flexible wire 19 and stent 16 are to be separated, the operation ring 20 is once pushed. Thereby, the flexible wire 19 is pushed forward. At this time, by the elastic deformation of the engaging member 42, the engaging plate 43 is disengaged from the side hole 41 of the stent 16. Further, as shown in FIG. 11B, the rear end portion of the engaging plate 43 is pulled out of the side hole 41 of the stent 16. At this time, the engaging plate 43 is restored to its straight original shape. In this state, the flexible wire 19 is pulled backward. Thereby, as shown in FIG. 11C, the engaging plate 43 is shifted beyond the side hole 41 of the stent 16 and is pulled out backward. Thus, the flexible wire 19 and stent 16 are separated, and the stent 16 is stayed at the stenotic part.

The apparatus with the above structure can achieve the following advantageous effects. In the present embodiment, the apparatus is set in the state in which the rear end portion of the engaging plate 43 of the engaging member 42 is inserted and hooked in the side hole 17a of the stent 16. Therefore, at the time of the procedure for positioning the stent 16 at the stenotic part of the bile duct, the stent 16 can be pulled back to the proximal end side by means of the flexible wire 19 even if the stent 16 is pushed too deeply.

When the flexible wire 19 and stent 16 are to be separated, the operation ring 20 is once pushed. Thereby, the flexible wire 19 is pushed forward. By the elastic deformation of the engaging member 42, the engaging plate 43 is disengaged from the side hole 41 of the stent 16, and in this state the flexible wire 19 is pulled backward. Thereby, as shown in FIG. 11C, the engaging plate 43 is shifted beyond the side hole 41 of the stent 16 and is pulled out backward. Thus, the flexible wire 19 and stent 16 are separated, and the stent 16 is positioned at the stenotic part.

Besides, in the present embodiment, the flexible wire 19 is connected over the stent 16 and pusher tube 21. Hence, the bending strength at the connection part between the stent 16 and pusher tube 21 is high. Even if the angle of bend of the curved part of the endoscope is large, buckling at the connection part between the stent 16 and pusher tube 21 can be reduced.

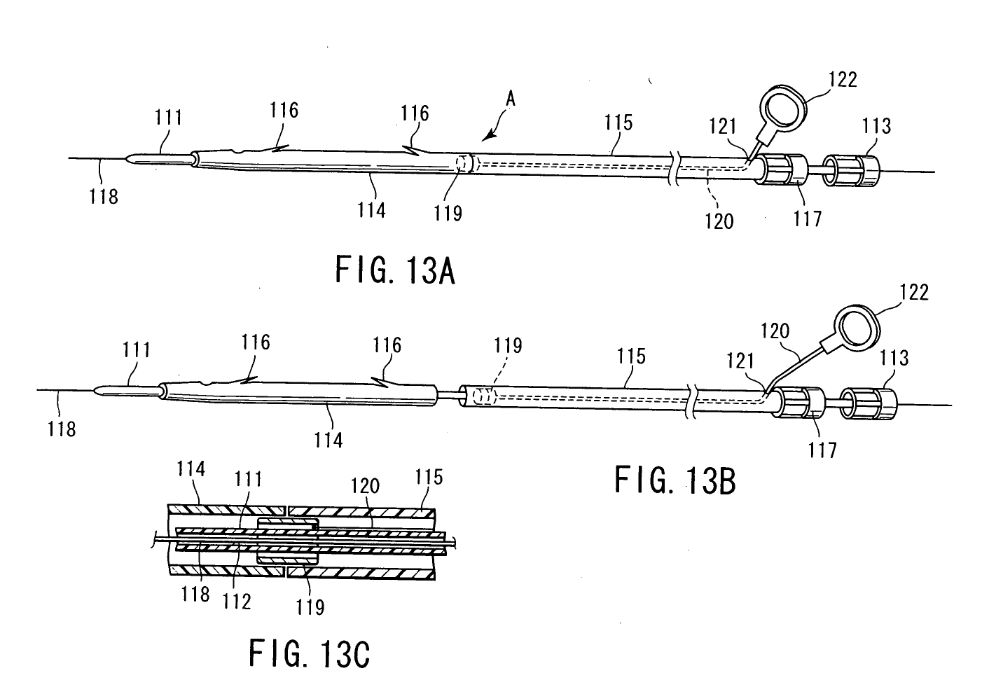

FIG. 13A to FIG. 13C show a stent delivery device according to an eighth embodiment of the present invention. As is shown in FIG. 13A, the stent delivery device according to the present embodiment is provided with an elongated guide catheter 111 that is insertable in a forceps channel of an endoscope (not shown). The guide catheter 111 is formed of a flexible synthetic resin material such as a fluoro-resin or a nylon resin. An inner cavity 112 is formed in the guide catheter 111 over the entire length thereof. A guide catheter cock 113 is provided near a proximal end portion of the guide catheter 111.

A stent 114, which serves as a stent, is provided on a distal end portion of the guide catheter 111 in the state in which the stent 114 is engaged with the guide catheter 111. A pusher tube 115 is provided on an outer peripheral surface of the guide catheter 111 on a proximal-end side of the stent 114. The pusher tube 115 is held in the state in which the pusher tube 115 is engaged with the guide catheter 111.

The stent 114 is a relatively soft hollow tube, which is formed of a high-polymer compound with biocompatibility, such as polyethylene or silicone rubber. It is desirable that the outer peripheral surface of the stent 114 be coated with a hydrophilic lubricant. Outer peripheral portions at both ends of the stent 114 are provided with mutually opposed flaps 116 for preventing removal.

The pusher tube 115 is formed of a flexible synthetic resin material such as a fluoro-resin or a nylon resin. A pusher tube cock 117 is provided at the proximal end of the pusher tube 21.

A guide wire 118 is axially passed through the inner cavity of the guide catheter 111. The guide wire 118 is an elongated metallic twisted wire. The distal end portion of the guide wire 118 is tapered. Further, the proximal end portion of the guide wire 118 is led out of the guide catheter cock 113.

As is shown in FIG. 13C, a cylindrical member 119 that serves as a connection mechanism is press-fitted in both the inner cavity of the stent 114 on the proximal end side thereof and the inner cavity of the pusher tube 115 on the distal end side thereof. The cylindrical member 119 separably connects the stent 114 and pusher tube 115. The cylindrical member 119 is formed of a synthetic resin material or a metallic material. The guide catheter 111 is passed through the inner cavity of the cylindrical member 119.

One end portion of an operation wire 120, which serves as release means, is connected to a proximal end portion of the cylindrical member 119. The other end portion of the operation wire 120 extends to the vicinity of the pusher tube cock 117 through the inner cavity of the pusher tube 115. A side hole 121 is formed in the pusher tube 115 in the vicinity of the pusher tube cock 117. The operation wire 120 is led out of the side hole 121 and connected to an operation ring 122.

The apparatus with the above structure can achieve the following advantageous effects. In the stent delivery device with the above-described structure, the stent 114 and pusher tube 115 are-connected by the cylindrical member 119. Thus, when the pusher tube 115 is axially moved, the stent 114 is also axially moved as one body. In addition, the cylindrical member 119 is press-fitted in the inner cavities of the stent 114 and pusher tube 115. Accordingly, the bending strength of the connection part between the stent 114 and pusher tube 115 is high. Hence, even if the angle of bend of the curved part of the endoscope is large, the connection part is not buckled and the stent 114 can be advanced to the target part.

If the operation wire 120 is pulled to the proximal end side by the operation ring 122 in the state in which the pusher tube 115 is held, the distal end portion of the cylindrical member 119 is removed from the inner cavity of the stent 114 and pulled into the inner cavity of the pusher tube 115. As a result, the stent 114 and pusher tube 115 are separated.

The operation of the eighth embodiment is described. When the stent delivery device according to this embodiment is used, the stent delivery device is set as follows.

To begin with, as shown in FIG. 13A, the operation wire 120 is inserted into the pusher tube 115. Then, a proximal end portion of the cylindrical member 119 is press-fitted in the inner cavity of the pusher tube 115 at the distal end portion thereof. The cylindrical member 119 is coupled to the operation wire 120.

Further, after the guide catheter 111 is passed through the pusher tube 115, the stent 114 is passed over the distal end portion of the guide catheter 111. In this state, the distal end portion of the cylindrical member 119 is press-fitted in the inner cavity of the stent 114, and the proximal end of the stent 114 is abutted upon the distal end of the pusher tube 115. Thereby, as shown in FIG. 13A, the pusher tube 115, stent 114 and cylindrical member 119 are set in the assembled state.

Thereafter, the insertion portion of the endoscope is inserted in the body cavity in advance, and a distal-end structural part, which is disposed at the distal end of the insertion portion of the endoscope, is guided to the vicinity of the bile duct.

Subsequently, the guide wire 118 is passed through the forceps channel of the endoscope that is inserted in the body. At this time, under observation using the endoscope and observation using X-rays, the guide wire 118 is advanced and the distal end portion of the guide wire 118 is guided into a stenotic part of the bile duct.

After the guide wire 118 is passed through the forceps channel of the endoscope, the guide catheter 111 on which the stent 114 and pusher tube 115 are set is passed over the guide wire 118. At this time, the guide catheter 111 is inserted into the forceps while being guided by the guide wire 118.

Subsequently, the guide catheter 111 and pusher tube 115 are advanced by a manual operation on the proximal end side of the guide catheter 111, and are led out of the distal-end structural part of the endoscope. In this state, the guide catheter 111 and stent 114 are inserted into the stenotic part. At this time, the stent 114 is engaged with the guide catheter 111, and the cylindrical member 119 is press-fitted in the inner cavities of the stent 114 and pusher tube 115. Thus, even if the guide wire 118 is curved with a large angle of bend, the stent 114 is not buckled. The stent 114 can be advanced together with the guide catheter 111 and pusher tube 115 and guided to the target part.

After the stent 114 is inserted into the stenotic part by means of the guide catheter 111, X-ray observation is performed. If it is confirmed by the X-ray observation that the stent 114 is pushed too deeply, the pusher tube 115 is pulled back to a position on the proximal end side. Thereby, the stent 114 can be pulled back by the cylindrical member 119, and the stent 114 can exactly be positioned at the target part.

Then, in the state in which the guide catheter 111 and guide wire 118 are left as such, the proximal end portion of the pusher tube 115 is held and the operation ring 122 is hooked with the finger and pulled toward the proximal end side. Thereby, the cylindrical member 119 is pulled into the inner cavity of the pusher tube 115 by the operation wire 120. As is shown in FIG. 13B, the distal end portion of the cylindrical member 119 is removed from the inner cavity of the stent 114, and the stent 114 and pusher tube 115 are separated. As a result, the stent 114 is stayed at the stenotic part.

In this case, the distal end portions of the guide catheter 111 and guide wire 118 are inserted into the stenotic part. Therefore, a contrast medium, etc. may be fed using the guide catheter 111 as a guide, or a subsequent treatment may be performed using the guide wire 118 as a guide.

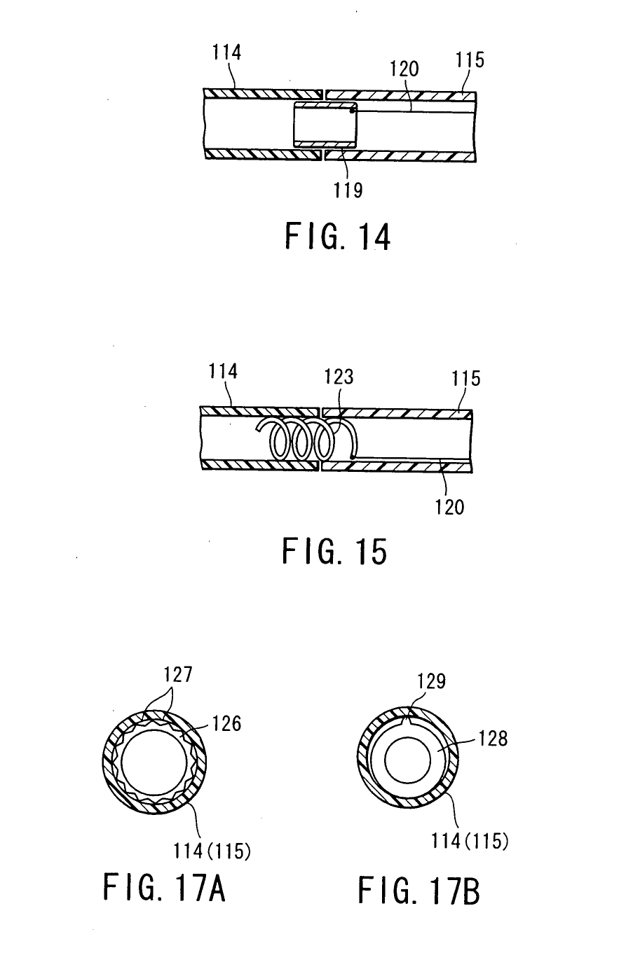

FIG. 14 shows a ninth embodiment of the invention. In this embodiment, the stent delivery device of the eighth embodiment (see FIG. 13A through FIG. 13C) is partly modified as follows. The parts common to those in the eighth embodiment are denoted by like reference numerals, and a description thereof is omitted.

In this embodiment, the guide catheter 111 of the eighth embodiment is not used. Accordingly, in this embodiment, the structure can be simplified and the cost can be reduced. Further, the stent 114 and pusher tube 115 can be made thinner. In a case where the stenotic part is small in size, the stent 114 can easily be inserted.

The advantageous effects of the ninth embodiment are the same as those of the eighth embodiment.

FIG. 15 shows a tenth embodiment of the present invention. In this embodiment, the guide catheter 111 is not used. In addition, a substantially cylindrical member that serves as a connection member is formed of a spiral member 123. A distal end portion of the spiral member 123 is press-fitted in the inner cavity of the stent 114, and a proximal end portion thereof is press-fitted in the inner cavity of the pusher tube 115. By using a spring member as the spiral member 123, the resilience force for restoring elastic deformation of the connection part becomes excellent and the anti-buckling property of the connection part can be improved.

The advantageous effects of the tenth embodiment are the same as those of the eighth embodiment.

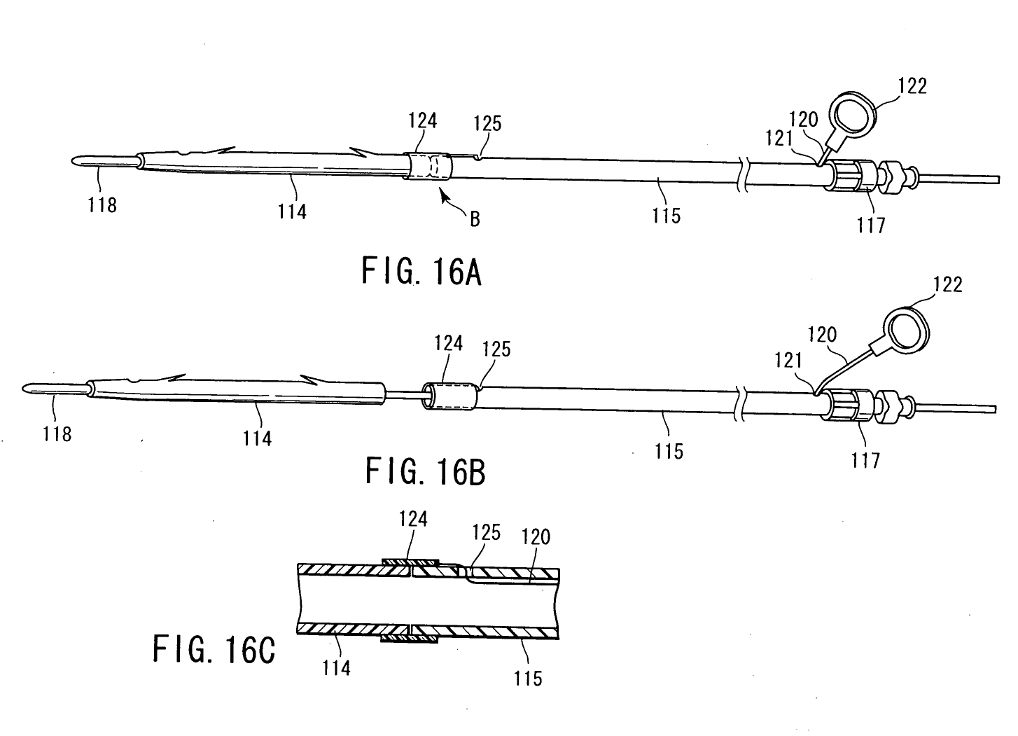

FIG. 16A to FIG. 16C show an eleventh embodiment of the present invention. In this embodiment, the guide catheter 111 is not used. A cylindrical member 124 is tightly fitted on the outer peripheral surfaces of the stent 114 and pusher tube 115.

A side hole 125 is formed in the side wall of the distal end portion of the pusher tube 115. The operation wire 120, which is coupled to the cylindrical member 124, is introduced into the inner cavity of the pusher tube 115 from the side hole 125.

Like the eighth embodiment, after the stent 114 is inserted into the stenotic part, X-ray observation is performed. If it is confirmed by the X-ray observation that the stent 114 is pushed too deeply, the pusher tube 115 is pulled back to a position on the proximal end side. Thereby, the stent 114 can be pulled back by the cylindrical member 124, and the stent 114 can exactly be positioned at the target part.

Then, the proximal end portion of the pusher tube 115 is held and the operation ring 122 is hooked with the finger and pulled toward the proximal end side. Thereby, the cylindrical member 124 is pulled onto the outer peripheral surface of the pusher tube 115 by the operation wire 120. As is shown in FIG. 16B, the distal end portion of the cylindrical member 124 is removed from the outer peripheral surface of the stent 114, and the stent 114 and pusher tube 115 are separated. As a result, the stent 114 is positioned at the stenotic part.

In this embodiment, there is no component in the stent 114 or pusher tube 115, and therefore the stent 114 and pusher tube 115 can be made thinner.

FIG. 17A shows a twelfth embodiment of the present invention. In this embodiment, the cylindrical member 119, which serves as the connection mechanism of the stent delivery device according to the eighth embodiment, is modified as follows.

A cylindrical member 126 of the present embodiment has an outer peripheral surface that is provided with a plurality of recess/projection portions 127 arranged in the circumferential direction thereof. Each recess/projection portion 127 extends in the axial direction of the cylindrical member 126. The cylindrical member 126 of this embodiment is press-fitted in the inner cavities of the stent 114 and pusher tube 115. In this case, the cylindrical member 126 is firmly press-fitted in the inner cavities of the stent 114 and pusher tube 115, and these parts are fixed.

FIG. 17B shows a modification of the cylindrical member 126 according to the twelfth embodiment. In this modification, as shown in FIG. 17B, a ridge portion 129 is axially provided on a part of the outer peripheral surface of a cylindrical member 128. The cylindrical member 128 of this embodiment is press-fitted in the inner cavities of the stent 114 and pusher tube 115. In this case, the cylindrical member 128 is firmly press-fitted in the inner cavities of the stent 114 and pusher tube 115.

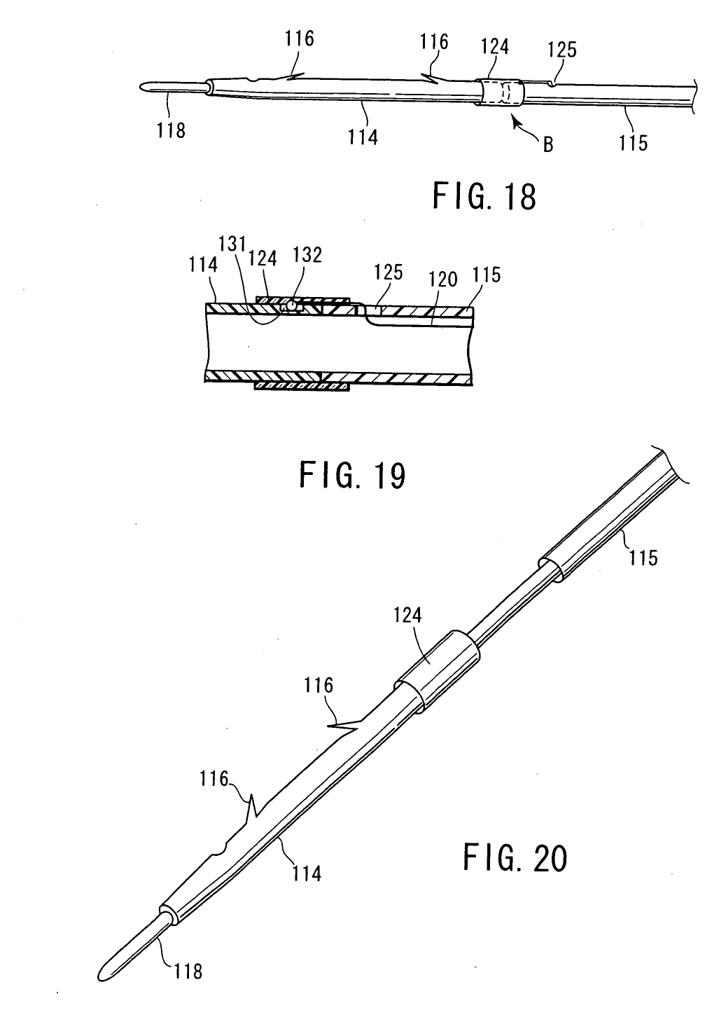

FIGS. 18 to 20 show a 13th embodiment of the invention. In this embodiment, the cylindrical member 124 of the eleventh embodiment (see FIGS. 16A to 16C) is formed of a heat-shrinkable tube. Further, as shown in FIG. 19, a recess portion 131 is formed at a part of the outer peripheral surface of the stent 114, which is covered with the cylindrical member 124. A ball chip 132 is embedded in the recess portion 131. The ball chip 132 is coupled to a distal end portion of the operation wire 120.

The heat-shrinkable tube is fitted in the state in which the ball chip 132 is placed in the recess portion 131 of the stent 114. Thus, the ball chip 132 is buried between the cylindrical member 124 and the recess portion 131 of the stent 114.

Next, the operation of the 13th embodiment is described. When the stent delivery device of this embodiment is to be used, the stent 114 and pressure tube 115 are coupled by the cylindrical member 124. Thus, when the pusher tube 115 is axially moved, the stent 114 is also axially moved as one body. In addition, the cylindrical member 119 is fitted over the outer peripheral surfaces of the stent 114 and pusher tube 115. Accordingly, the bending strength of the connection part between the stent 114 and pusher tube 115 is high. Hence, even if the angle of bend of the curved part of the endoscope is large, the connection part is not buckled and the stent 114 can be advanced to the target part.

If the operation wire 120 is pulled to the proximal end side by the operation ring 122 in the state in which the pusher tube 115 is held, the ball chip 132 is pulled and removed from between the cylindrical member 124 and the recess portion 131 of the stent 114. With the removal of the ball chip 132, as shown in FIG. 20, the pusher tube 115 is pulled out of the inner cavity of the cylindrical member 124, and the stent 114 and pusher tube 115 are separated. Thereby, the stent 114 is positioned in the stenotic part.

With the present embodiment, too, the same advantageous effects as in the eleventh embodiment can be obtained. In addition, in this embodiment, in particular, when the stent 114 and pusher tube 115 are separated, the cylindrical member 124 remains attached to the stent 114 that is separated from the pusher tube 115. Therefore, when the stent 114 is changed, the part of the cylindrical member 124 attached to the stent 114 can be held, and this facilitates the work for removing the stent 114.

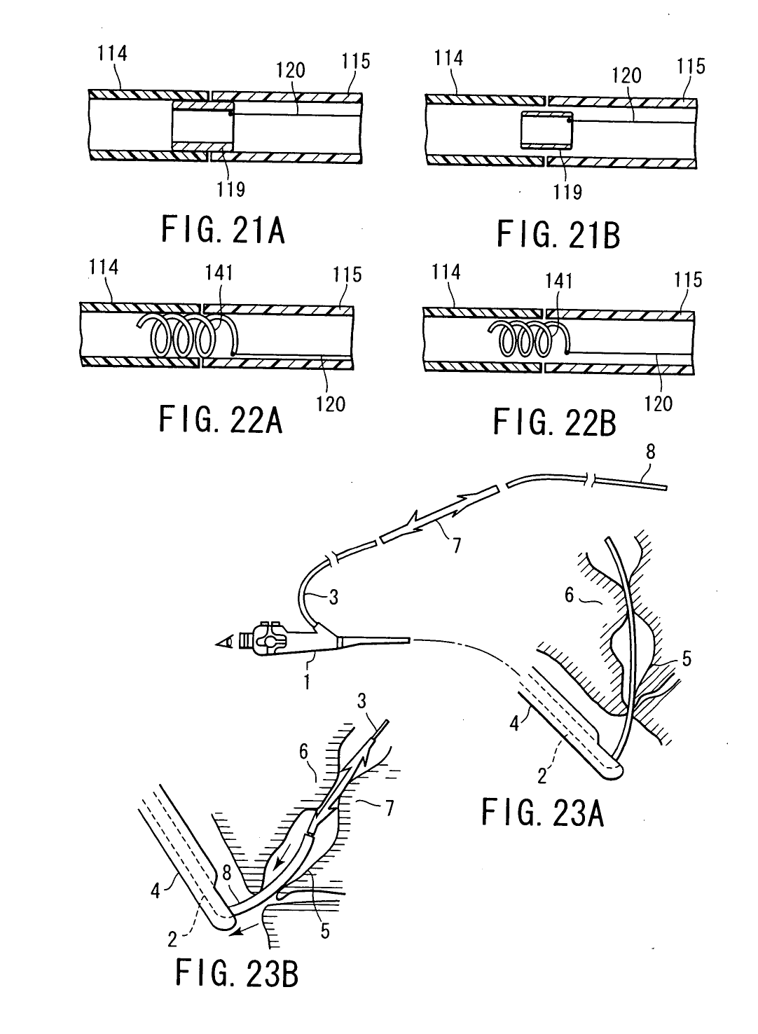

FIG. 21A and FIG. 21B show a 14th embodiment of the present invention. In this embodiment, the cylindrical member 119 according to the eighth embodiment (see FIG. 13A to FIG. 13C) is formed of a shape-memory alloy tube.

For example, at a normal temperature (reference temperature), the shape-memory alloy tube of the cylindrical member 119 is broadened to have a greater outside diameter than the stent 114, as shown in FIG. 21A. At this time, the stent 114 and pusher tube 115 are separably coupled by the cylindrical member 119.

On the other hand, for example, when the shape-memory alloy tube of the cylindrical member 119 is heated up to a higher temperature than the reference temperature or cooled down to a lower temperature than the reference temperature, the shape-memory alloy tube deforms to have a less outside diameter than the stent 114, as shown in FIG. 21B.

In the present embodiment, if the shape-memory alloy tube of the cylindrical member 119 is deformed to a reduced shape, the stent 114 and pusher tube 115 can be separated.

The shape-memory alloy tube of the cylindrical member 119 may be configured to be heated by application of electric power.

FIG. 22A and FIG. 22B show a modification of the 14th embodiment. In this modification, the shape-memory alloy tube of the cylindrical member 119 according to the 14th embodiment (see FIG. 21A and FIG. 21B) is replaced with a coil-shaped engaging member 141. This engaging member 141 is formed of a spiral member of a shape-memory alloy.

For example, at a normal temperature (reference temperature), the shape-memory alloy of the engaging member 141 is broadened to have a greater outside diameter than the stent 114, as shown in FIG. 22A. At this time, the stent 114 and pusher tube 115 are separably coupled by the engaging member 141.

On the other hand, for example, when the shape-memory alloy of the engaging member 141 is heated up to a higher temperature than the reference temperature or cooled down to a lower temperature than the reference temperature, the shape-memory alloy deforms to have a less outside diameter than the stent 114, as shown in FIG. 22B.

In the present embodiment, if the shape-memory alloy of the engaging member 141 is deformed to a reduced shape, the stent 114 and pusher tube 115 can be separated.

As has been described above, the present invention is effective in the technical field of a stent delivery device that is used in performing an operation for inserting and positioning a stent in a body cavity using an endoscope, and in the technical field of the manufacture and use of this stent delivery device.

1. A stent delivery device comprising:
a stent;
a guide member having an inner cavity, at least a distal end portion of the guide member being insertable in the stent;
a pulling member including an engaging portion that is inserted in a gap between the inner cavity of the stent and the guide member and detachably engages the stent, and an insertion portion that passes through at least a part of the inner cavity of the guide member, the pulling member executing an operation for pulling the stent when the engaging portion engages the stent; and
an engagement-releasing member that moves the pulling member in an axial direction of the guide member, thereby releasing the engagement between the engaging portion and the stent.

2. The stent delivery device according to claim 1, wherein the guide member includes a pusher tube having an outside diameter that is greater than an inside diameter of the stent.

3. The stent delivery device according to claim 2, wherein the stent is provided with a large-diameter portion on a proximal end side of the stent, the large-diameter portion having an outside diameter that is greater than an inside diameter of the stent.

4. The stent delivery device according to claim 2, wherein at least a part of a proximal end side portion of the pulling member is located in an inner cavity of the pusher tube.

5. The stent delivery device according to claim 2, wherein the pulling member includes a proximal-end operation portion that is situated at a proximal end portion of the pusher tube and is movable in an axial direction of the pusher tube.

6. The stent delivery device according to claim 1, wherein a plurality of said pulling members are situated within the stent.

7. The stent delivery device according to claim 1, wherein the engaging member includes at least one of a curved portion, a spiral portion and a folded portion within the stent.

8. The stent delivery device according to claim 1, wherein the stent has at least one side hole, and
the engaging member has spring elasticity and is disposed through the side hole of the stent and a gap between the inner cavity of the stent and the guide member.

9. A stent delivery device comprising:
a stent;
a pusher tube having an inner cavity and an outside diameter that is greater than an inside diameter of the stent;
a substantially cylindrical connection member having a distal end portion located at a proximal end portion of the stent, and a proximal end portion located at a distal end portion of the pusher tube, the connection member separably coupling the stent and the pusher tube;
a pulling member having a distal end portion connected to a proximal end of the connection member, and at least a portion passed through the inner cavity of the pusher tube; and
an engagement-releasing member that releases an engagement state of the stent by an operation of axially moving the pulling member.

10. The stent delivery device according to claim 9, further comprising a guide member having an inner cavity, at least a distal end portion of the guide member having such an outside diameter as to be insertable in an inner cavity of the stent.

11. The stent delivery device according to claim 9, wherein the connection member is press-fitted on an inner periphery or an outer periphery of the stent at a distal end portion of the connection member, and the connection member is freely passed over an inner periphery or an outer periphery of the pusher tube at a proximal end portion of the connection member.

12. The stent delivery device according to claim 9, wherein the connection member has at least one projection portion on an outer peripheral surface thereof, which is press-fitted in an inner cavity of each of the stent and the pusher tube.

13. The stent delivery device according to claim 9, wherein the connection member is a spiral member with spring characteristics.

14. The stent delivery device according to claim 9, wherein the connection member is coupled to the stent, and the engagement-releasing member removes the connection member from the pusher tube.

15. The stent delivery device according to claim 9, wherein the connection member includes a press-fitting portion that is press-fitted in an inner cavity of each of the stent and the pusher tube, and
the press-fitting portion is deformable between a first shape that has such a first outside diameter as to enable engagement with the stent and the pusher tube, and a second shape that has a second outside diameter, which is different from the first outside diameter, when the engagement with the stent is released.
